(19) United States
(12) Patent Application Publication
(10) Pub. No.: US 2024/0408217 A1
(43) Pub. Date: Dec. 12, 2024

(54) POLYMER-DRUG CONJUGATE SYSTEMS FOR MONTELUKAST TO AVOID ITS PSYCHOTIC SIDE EFFECTS

(71) Applicants: Sayyed lbrahim Shah, Mardan (PK); Muhammed Raza Shah, Karachi (PK); Long Chau Ming, Mardan (PK); Ismail Shah, Mardan (PK); Shafi Ullah, Karechi (PK)

(72) Inventors: Sayyed lbrahim Shah, Mardan (PK); Muhammed Raza Shah, Karachi (PK); Long Chau Ming, Mardan (PK); Ismail Shah, Mardan (PK); Shafi Ullah, Karechi (PK)

(21) Appl. No.: 18/206,638

(22) Filed: Jun. 7, 2023

(51) Int. Cl. A61K 47/61; A61K 31/47; A61K 47/54

(52) U.S. Cl. CPC A61K 47/61; A61K 31/47; A61K 47/543

(57) ABSTRACT

The present invention relates to a polymer-drug conjugate comprised of the active (Montelukast) coupled directly to polymers (Polyethyleneimine and/or Chitosan), wherein the carrier substance is substantially resistant to permeation through the blood-brain barrier (BBB) under ambient conditions. The coupling between the active and the carrier substance is such that said coupling is not cleaved/substantially stable under said ambient conditions, so as to prevent/reduce the entry of the active into the brain. The present invention also relates to a composition including such a conjugate and the processes of preparing this conjugate and composition.

### BACKGROUND OF THE INVENTION

### 1. Field of the Invention

This invention generally relates to pharmaceutical composition and method of preparation thereof of polymer drug-conjugates. The invention more specifically relates to the technical field of biomedicine, in particular to a Polymer Drug-conjugate of the therapeutically active (Montelukast) and polyethyleneimine (PEI) and/or Chitosan polymer.

### 2. Description of Related Art

Montelukast is a leukotriene inhibitor and is used for the symptomatic treatment of asthma and allergic rhinitis (by preventing the bronchoconstriction) in adults, as well as its given orally—and, is an effective initial preventive therapy for children with mild asthma (Hon et al., 2014; Paggiaro and Bacci, 2011; Yilmaz et al., 2013). There have been reports of neuropsychiatric events (Glockler-Lauf et al., 2019) and it was on Mar. 4, 2020 when the FDA issued a “Black box warning” indicating serious mental health issues associated with the use of the drug, i.e. Montelukast. (www.fda.gov.).

For any compound to show its effect (whether therapeutic or not) on the CNS needs to cross the blood-brain barrier (BBB) which is a membrane that selectively allows the movement of the compounds across it. One of the major factors affecting the ability to permeate through the BBB is the molecular weight (molecular weight cut off up to 600 Da) (Banks, 2009).

Reports of the montelukast permeating through the BBB and its unwanted neuropsychiatric side effects have been published (www.mentalhealthexcellence.org). Here, conjugation of the drug (montelukast) with a polymer (PEI) via a non-biodegradable bond is proposed based upon the hypothesis that this conjugation (with the polymer backbone) will prevent the permeation of the free drug across the BBB by virtue of its molecular weight. Currently, there is one polymer conjugate in the market (Naloxegol, PEG-Naloxone conjugate) to overcome opioid-induced constipation owing to the same proposed mechanism (Floettmann et al., 2017), thus providing a proof of concept for this study.

### SUMMARY OF THE INVENTION

The present invention relates to a polymer-drug conjugate comprised of the active (Montelukast) coupled directly to polymers (Polyethyleneimine and/or Chitosan), wherein the carrier substance is substantially resistant to permeation through the blood-brain barrier (BBB) under ambient conditions.

The coupling between the active and the carrier substance is such that said coupling is not cleaved/substantially stable under said ambient conditions, so as to prevent/reduce the entry of the active into the brain. The present invention also relates to a composition including such a conjugate and the processes of preparing this conjugate and composition.

### BRIEF DESCRIPTION OF DRAWINGS

FIG. 1 represents the ¹H-NMR spectrum of montelukast.

FIG. 2 shows ¹H-NMR spectrum of PEI-montelukast conjugate.

FIG. 3 shows stability of PEI-montelukast conjugate in rat plasma.

FIG. 4 is the Cell viability assay results of PEI-montelukast conjugate.

FIG. 5 is the IL-1β production in AMs cells after incubation with montelukast and PEI-montelukast conjugate for 24 h.

FIG. 6 is the TNF-α production in AMs cells after incubation with montelukast and PEI-montelukast conjugate for 24 h.

FIG. 7 shows the concentration of free montelukast found in the brain of the rats.

### DETAILED DESCRIPTION OF THE INVENTION

The present invention relates to a polymer-drug conjugate system based on the covalent coupling of montelukast to a carrier substance (PEI and/or Chitosan), where the carrier substance is substantially resistant to permeation through the blood-brain barrier (BBB).

The present invention has utility in respiratory diseases, especially asthma and chronic obstructive pulmonary disorder (COPD), whereby the selected active (montelukast) is stable in a conjugate or composition as used by the user.

According to a first embodiment of the present invention, there is provided with a conjugate suitable to be used orally/IV route, the conjugate comprises active coupled directly to the carrier substance, wherein the carrier substance is substantially resistant to the permeation through BBB under ambient conditions and wherein the coupling between the active and the carrier substance is such that said coupling is not cleaved/substantially resistant cleaving under said ambient conditions when said conjugate is used by the user.

According to a second embodiment of the present invention, there is provided a composition suitable to be used orally/IV route, the composition comprising a conjugate and at least one inert diluent or excipient therefore, said conjugate comprising of active coupled directly to a carrier substance (PEI and/or Chitosan), wherein the carrier substance is substantially resistant to permeation through the BBB of a user under ambient conditions, and wherein the coupling between the active and the carrier substance is such that said coupling is not cleaved/substantially resist cleaving under said ambient conditions when said conjugate has been used by the user.

Also, according to the second embodiment of the present invention, there is provided with a method of testing the stability of the conjugate, wherein said method comprises of:

Testing the stability in vitro upon incubation with rat plasma to probe stability under physiological conditions.

The term “active” as referred to herein denotes montelukast.

The term “ambient conditions” as referred to herein denotes the physiological conditions to which the conjugate is exposed after the administration of the said conjugate by the user through an oral route.

The terms “coupled” or “coupling” as used herein denote a direct attachment between the active and the carrier substance. Thus, the active can be directly attached to the carrier substance based on a suitable bonding there between as hereinafter described in greater detail.

The term “carrier substance” as used herein denotes a substance which is substantially resistant to permeation through the BBB of a user under ambient conditions. For example, if a carrier substance of sufficient molecular weight is selected, there will be little permeation through the BBB of a user. Additionally, or as an alternative proposal, the carrier substance can be selected to be sufficiently hydrophilic, which should increase resistance to permeation through the BBB by the carrier substance.

For an agent to produce any effect in the brain or CNS, whether therapeutic or not, it must pass through the BBB. Generally, high molecular weight and/or hydrophilic agents will exhibit low permeation through the BBB. In the present case, the molecular weight of the carrier substance is selected to provide the required resistance to BBB permeation.

Such reduction in BBB permeation is beneficial in achieving a corresponding reduction in any side effects associated as carrier substances of sufficient molecular weight permeate very poorly through BBB as explained above.

The active used in the present invention includes at least one group that forms an amide bond with at least one group present in the carrier substance (Polymer). Active used in the present invention includes at least one OH group in its structure, whereby the active is thus particularly suited to form an amide bond with one or more amine groups as present in a carrier substance as used in the present invention. Such a resulting amide bond is stable at ambient conditions and do not hydrolyze/result in minimum hydrolysis.

The carrier substance to which the active is coupled may be a single substance, or a mixture of different substances, all of which may bond with the active. A particularly preferred carrier substance comprises one or more polymers, all of which may bond with the active. Such polymers are preferably selected so as to provide the desired molecular weight so as to provide resistance to BBB permeation as herein before described. Alternatively, the hydrophilicity of the carrier substance, preferably a polymer, may be selected so as to provide resistance to BBB permeation.

Suitable molecular weights of polymers used in the present invention to provide the desired resistance to BBB permeation are usually greater than 1000 daltons, as measured by size exclusion chromatography, also commonly known as gel permeation chromatography. Typically, however, the molecular weights of polymers used in the present invention are less than 1.8 million daltons again as measured by size exclusion chromatography. Preferred molecular weight ranges can be 2,000 to 50,000 daltons, or 10,000 to 300,000 daltons, for a polymer to be used in the present invention. These molecular weights are number average molar mass.

Even more preferably, the present invention thus provides conjugates and compositions wherein an active as described herein is directly coupled to a polymer carrier substance by way of an amide linkage, which can, in use, be advantageously stable at ambient conditions.

The preferred polymer carrier substances used in the present invention are selected from the group consisting polyethyleneimine, chitosan, other suitable polymers with an amine group, and mixtures thereof.

Polyethyleneimine and/or chitosan have the additional benefit of being well-established biodegradable polymers. This is particularly beneficial in preventing injury to the lungs upon long-term exposure to the said polymers.

Coupling the active and carrier substance is performed by many different reactions that are well known.

One approach is the functionalization of either amine or OH groups. For example, by an acylating amine, or OH with succinic anhydride, e.g., in pyridine followed by the reaction with the nucleophilic (ROH or RNH2) in the presence of a suitable coupling reagent. Alternatively, the amine group of the polymeric carrier can react with the O-succinate i.e. RO—COCH2CH2COOH in the presence of DIPEA/DIC.

The above reaction steps are preferably carried out in the presence of a coupling agent. The reaction can preferably use PyBop as a coupling agent (PyBop is the common abbreviation for benzotriazol-1-yl-oxytripyrrolidinophosphonium hexafluorophosphate). Other suitable coupling agents that could be used in these reactions include benzotriazol-1-yloxy) tris (dimethylamino) phosphonium hexafluorophosphate which is commonly known as BOP or 3-(diethoxyphosphoryloxy)-1,2,3-benzotriazin-4 (3H)-one which is commonly known as DEPBT or 1-[bis (dimethylamino) methylene]-1H-1,2,3-triazolo[4,5-b]pyridinium 3-oxide hexafluorophosphate which is commonly known as HATU or 2-(1H-benzotriazol-1-yl)-1,1,3,3-tetramethyluronium hexafluorophosphate which is commonly known as HBTU. The above reaction steps are also preferably carried out in the presence of DIPEA (N,N-diisopropylethylamine, also commonly known as Hünig's base) as well as carbonyldiimidazole (DIC).

The above reaction steps are also preferably carried out in the presence of a polar aprotic solvent. DMSO (dimethyl sulphoxide) is a preferred such solvent, but other suitable solvents can also be used and could include, for example, dimethylformamide, dimethylacetamide or N-methyl-2-pyrrolidone.

The reaction is typically performed at room temperature, or at slightly elevated temperatures, such as 35° C. The temperature of the reaction, as well as the precise coupling agent, solvent and base chosen affect the time needed to allow the reagents to react, but the reaction is typically completed within 24 hours.

The present invention is now further illustrated by the following examples, which are not intended to limit the scope of the invention in any way.

### Example 1

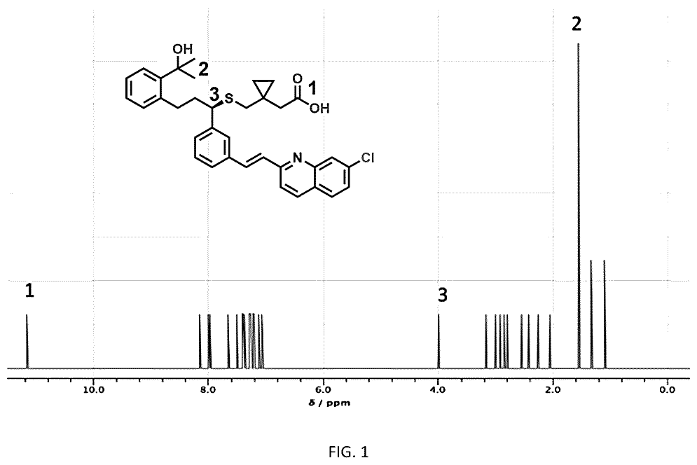

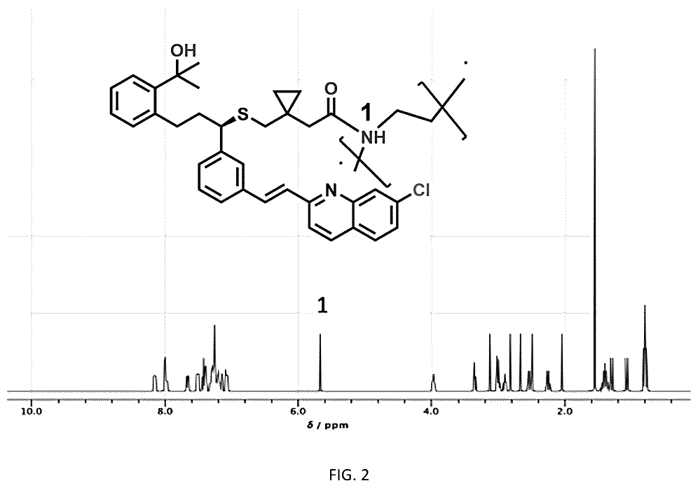

DOWEX ion exchange resin (12.5 g) was washed three times with 250 ml aliquots of distilled water. To this 0.1 g of montelukast-sodium was passed through the resin. 500 mg of montelukast was dissolved in 60 mL dimethyl sulphoxide (DMSO). 0.5 ml of N, N-diisopropylethylamine (DIPEA) and 0.9 g of benzotriazol-1-yl-oxytripyrrolidinophosphonium hexafluorophosphate (PyBop) were added and left stirring at room temperature for 1 hour. 1 g of polyethyleneimine (PEI) was then added and the mixture left stirring for a further 24 hours at room temperature. The resultant PEI-montelukast conjugate was then recovered from the reaction mixture by dialysis against ethanol by using a dialysis membrane of molecular weight cut off 1000 Da. FIG. 1 shows the 1H NMR spectrum of pure montelukast whereas, FIG. 2 is the 1H NMR spectrum of PEI-montelukast conjugate.

### Example 2

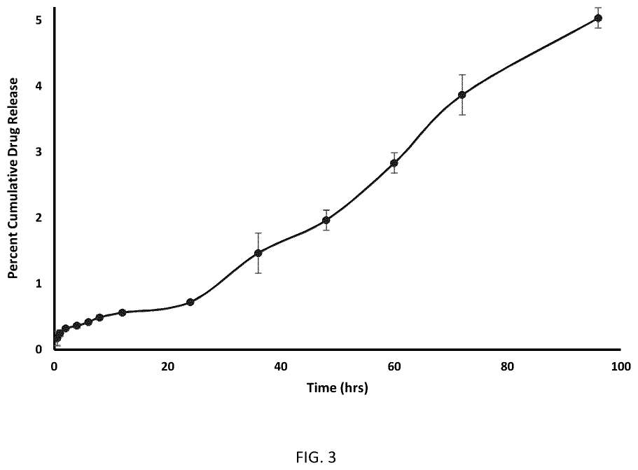

Stability studies of PEI-montelukast were carried out in rat plasma. Briefly, 100 μL of PEI-montelukast conjugate solution was added to 400 μL of rat plasma, before vertexing for 3 mins to ensure complete mixing. The samples were incubated at 37° C. to mimic physiological temperatures. After that, the samples were quenched by adding 20 μl of methanol. This was then vortexed for 1 min, followed by centrifugation at 4500 rpm for 1 min. This supernatant was removed and filtered through a 0.45 μm syringe filter. The filtered supernatant was then analysed via HPLC. This analysis showed that the PEI-montelukast conjugates was stable in plasma as shown in FIG. 3.

### Example 3

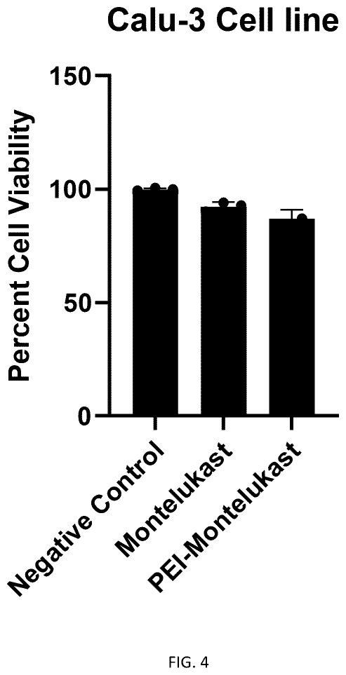

Calu-3 cells were seeded into 96-well plates at a density of 1×105 cells/mL, suspended in 100 μL of complete medium, and then incubated for 24 h. After incubation, the supernatant was aspirated from all wells and replaced with 100 μl of test or control sample in fresh medium, together with 50 μl of a 5 mg/mL MTT solution. The cells were then incubated at 37° C. under 5% CO2 for 4 h. Thereafter, the media and MTT were aspirated and 100 μL of DMSO was added. The absorbance was then measured at a wavelength of 570 nm using a microplate reader. The percentage of viable cells was calculated and compared with the percentage in the negative control. The results are shown in FIG. 4 indicating the PEI-montelukast conjugate was non-toxic in the tested cell line.

### Example 4

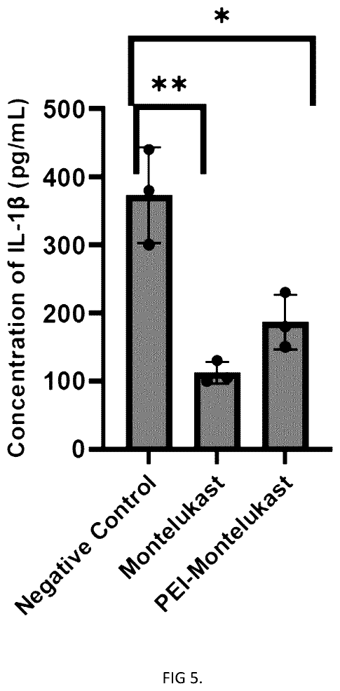

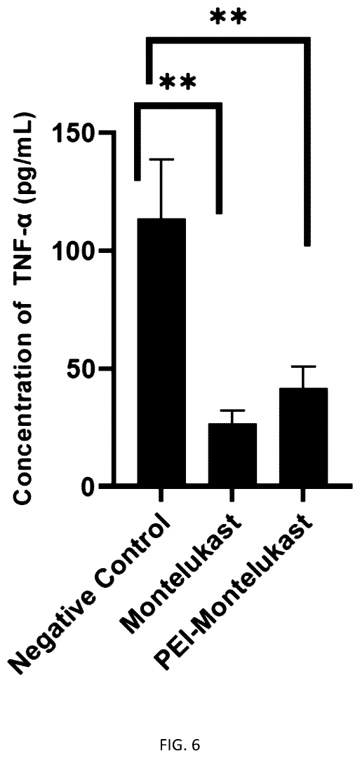

NR8383 cells were seeded at a 7.7×105 cells/mL concentration into 96 well plates containing the medium. After 24h of incubation, 100 μl of the sample or 1000 ng/ml of the positive control, LPS, in fresh medium, was added to each well. Wells with media only served as negative controls. Supernatants were collected and assayed for IL-1β and TNF-α using a rat ELISA kit. The results are shown in FIG. 5 and FIG. 6 indicating the PEI-montelukast conjugate produced nearly similar IL-1ß and TNF-α inhibitory effects in comparison with free montelukast.

### Example 5

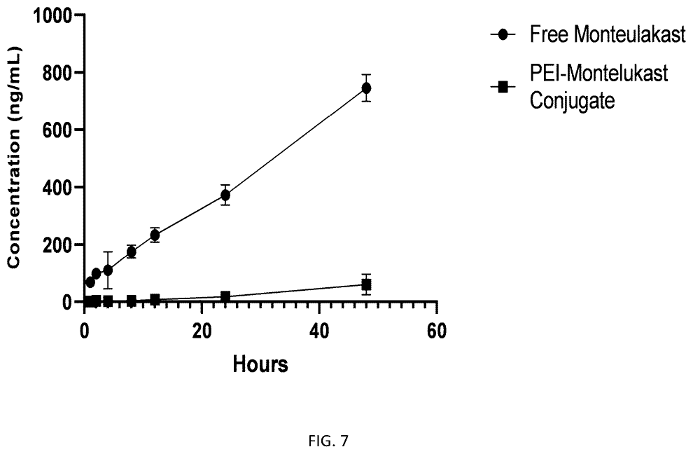

Rat brains were homogenised with an equal weight of water. Aliquots of homogenate from each sample were transferred to a 96 well plate and 3 volumes of acetonitrile containing an internal standard (200 ng/ml tolbutamide) were added to each. After centrifugation (15 min at 1500 g), supernatant (50 uL) was transferred to another 96 well plates and 100 μl of water was added. Control brain tissue was treated in the same way to provide blank samples and calibration standards by spiking control brain homogenate with known amounts of montelukast. The results are shown in FIG. 7 indicating less concentration of the drug fro the PEI-montelukast conjugate in comparison with free montelukast.

1. A carrier drug conjugate system comprising montelukast coupled directly to a carrier substance, wherein the carrier substance is polyethyleneimine and/or Chitosan.

2. A composition of the carrier drug conjugate suitable for oral/IV administration to human wherein the system is made of 1 g of polyethyleneimine (PEI) and/or Chitosan, 500 mg montelukast and at least one inert diluent such as sterile/water for injection.

3. The carrier drug conjugate system is coupled through an amide bond between the active and the carrier system, where the said conjugate is sufficiently stable under ambient conditions, and wherein the said conjugate comprising of an active coupled directly to a carrier substance, where the carrier substance is substantially resistant to permeation through the Blood-Brain Barrier (BBB) under ambient conditions.

4. The carrier drug conjugate system claimed in claim 1 is providing symptomatic relief in conditions like Asthma, Chronic Obstructive Pulmonary Disorder (COPD).

5. The carrier drug conjugate system composition as claimed in claim 2 possess substantial inhibitory activity against the inflammatory modulators, e.g., TNF-α and IL-β.

6. The carrier drug conjugate system composition as claimed in claim 2 is safe, non-toxic and biocompatible.
